(19) United States
(12) Patent Application Publication
(10) Pub. No.: US 2023/0181972 A1
(43) Pub. Date: Jun. 15, 2023

(54) ENCLOSURES FOR RUNNING PERFORMANCE ANALYSIS

(71) Applicant: Harry L. Reynolds, Jr., Columbus, OH (US)

(72) Inventor: Harry L. Reynolds, Jr., Columbus, OH (US)

(21) Appl. No.: 17/924,737

(22) PCT Filed: May 10, 2021
(86) PCT No.: PCT/US2021/031550

§ 371 Date: Nov. 11, 2022

Related U.S. Application Data
(60) Provisional application No. 63/022,915, filed on May 11, 2020.

(51) Int. Cl. A63B 24/00; A63B 69/00; A63B 71/06

(52) U.S. Cl. CPC A63B 24/0062; A63B 69/0028; A63B 71/0622; A63B 2071/0625; A63B 2024/0068; A63B 2071/0638

(57) ABSTRACT

The present disclosure provides a system for determining running performances using an enclosure. The system includes a running surface within an enclosure. The system can also include sensors, audio devices, display devices, and lighting devices along the length of the running surface. The system can further include user-wearable devices having mobile sensors. Additionally, the system can include a processor and a storage device storing program instructions that control the system to trigger a running performance and log data of the running performance received from the sensors and the mobile sensors. Additionally, the system can generate audiovisual cues using the audio, display, and lighting devices.

### BACKGROUND

Running is a fundamental element of many competitive sports. Proper running technique can substantially improve an athlete's performance and prevent injuries. To improve the athlete's technique, a running coach may assess various factors of the athlete's motion, such as stride rate, ground contact time, bounce, and protonation. Having more complete information of the athlete's performance permits the coach to better analyze the athlete's running technique and determine improvements. It would therefore be desirable to provide for a system that increases the amount of information collected from running performances.

### SUMMARY

The following presents a simplified summary of the disclosed subject matter in order to provide a basic understanding of some aspects of the disclosed subject matter. This summary is not intended to identify key or critical elements of the disclosed subject matter or delineate the scope of the claimed subject matter.

The present disclosure provides structures, systems, and methods for determining running performances. A system in accordance with the present disclosure can include a running surface within an enclosure. The system can also include sensors, audio devices, display devices, and lighting devices along the length of the running surface. The system can further include wearable devices having mobile sensors. Additionally, the system can include a processor and a storage device storing program instructions that control the system to perform operations including triggering a running performance and logging data of the running performance received from the sensors and the mobile sensors. Additionally, the operations can include generating audiovisual cues using the audio, display, and lighting devices.

### DRAWINGS

FIG. 1 shows a system block diagram illustrating an example of an environment for a system in accordance with aspects of the present disclosure.

FIG. 2 shows a front view of illustrating the example of a system in accordance with aspects of the present disclosure.

FIG. 3 shows a sectional side perspective view illustrating the example of the system in accordance with aspects of the present disclosure.

FIG. 4 shows a block diagram illustrating an example of a computing system in accordance with aspects of the present disclosure.

FIG. 5 shows a flow block diagram illustrating an example of a process for transporting an enclosure in accordance with aspects of the present disclosure.

FIG. 6 shows a flow block diagram illustrating an example of a process for determining a running performance in accordance with aspects of the present disclosure.

### DETAILED DESCRIPTION

The present disclosure relates to evaluating and teaching of running techniques. More specifically, the present disclosure relates to capturing information about users' running form to improve running technique.

Implementations of structures, systems and methods disclosed herein automatically capture information of users' running technique and provide performance cues. Additionally, implementations disclosed herein provide a user-friendly, entertaining, and motivational training system permitting athletes to the users to capture solo running sessions without assistance from a provider or a separate operator of the system.

As detailed herein, implementations consistent with the present disclosure provide a system including an instrumented enclosure for capturing detailed information of the users' technique from running performances while allowing the users to run in in a natural and untethered manner, as they would in an open race track. The enclosure can be a closed environment including a running surface and sensors that capture the user's running performance. The sensors can gather data related to the user's motion, mechanics, and physical state for analysis by a trainer or coach. For example, in some implementations, the instrumentation can include a series of cameras mounted in locations along the length of the running surface that capture images of user form from a variety of perspectives and angles as they run through the enclosure. Also, the sensors can include wearable devices that capture information describing the user's foot strikes on the running surface and collect biometric data describing the user's physical state. Further, the sensors can include motion and environmental sensors for detecting events and conditions within the enclosure.

In some implementations, the enclosure can also include cueing devices, such as display, lighting, and audio devices, that provide the user with motivation and feedback. The cueing devices can use active and passive components. For example, in some implementations, the lighting devices can provide reference markers detectable by the cameras. In some implementations, the cuing devices can generate sound and images indicating a target running pace, tempo, and form for the user. In some implementations, the cuing can be an image or indicator moving along the length of the enclosure indicating a desired pace displayed using the lighting or display devices. Further, in some implementations, the cueing can be time-synchronized recordings of previous running performances by the user or others displayed using the display devices. Also, the instrumentation can include passive or active distance markers indicating reference distances and positions to the user.

Reference will now be made in detail to specific implementations illustrated in the accompanying drawings. In the following detailed description, numerous specific details are set forth to provide a thorough understanding of the disclosed implementations. However, it will be apparent to one of ordinary skill in the art that implementations may be practiced without these specific details. In other instances, well-known methods, procedures, components, circuits, and networks have not been described in detail so as not to unnecessarily obscure aspects of the implementations.

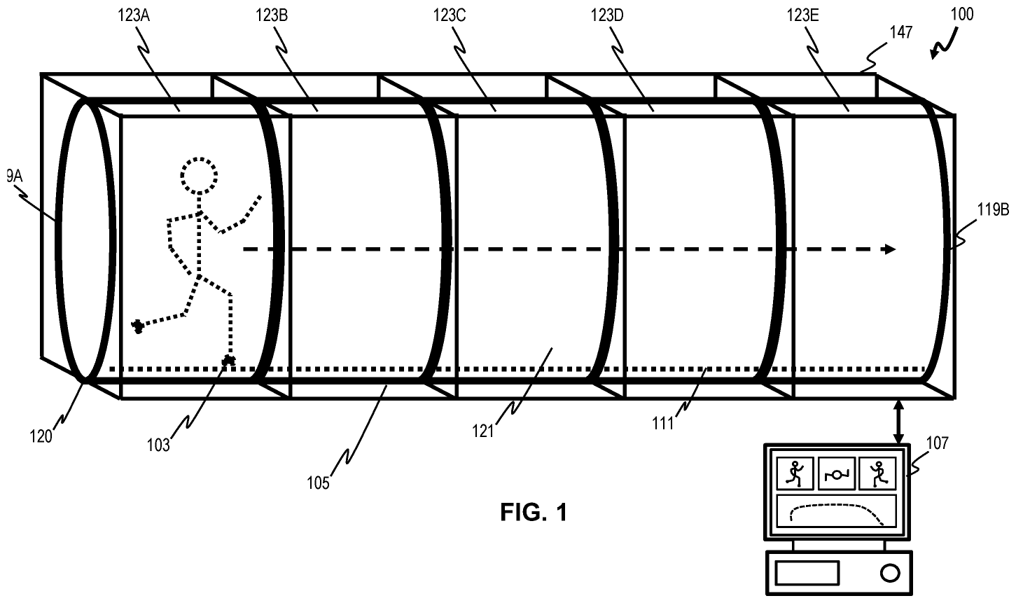

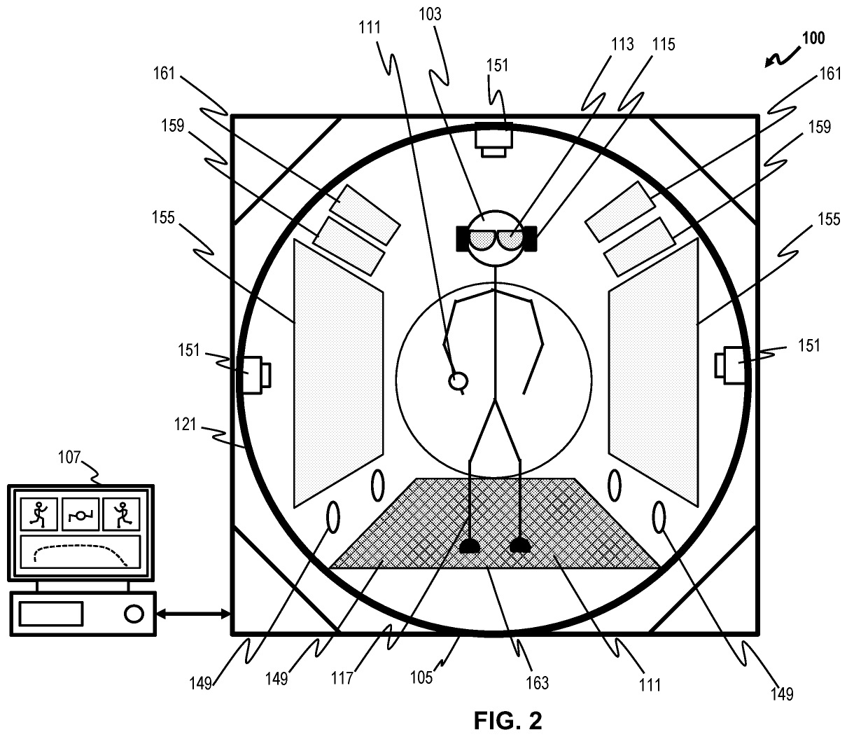

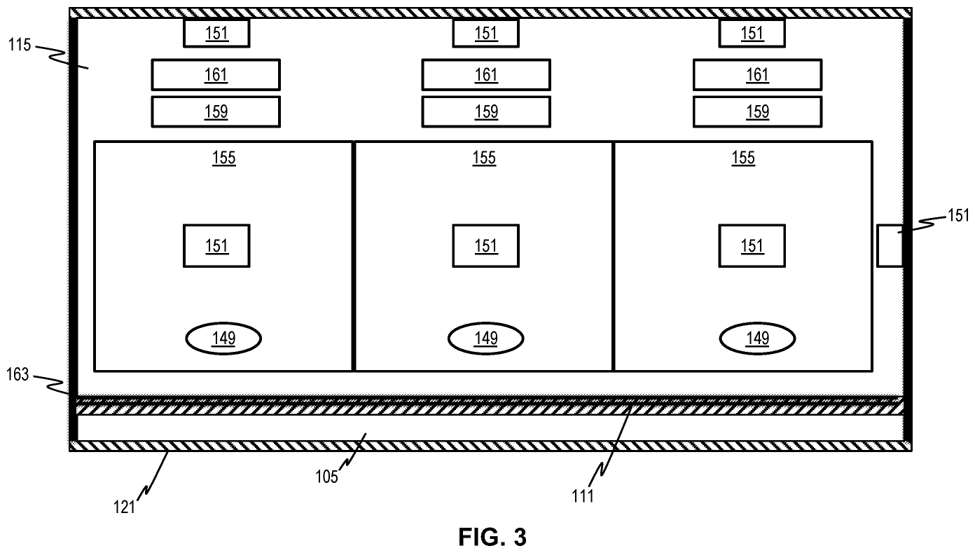

FIG. 1 shows a system block diagram illustrating an example environment of a system 100 in accordance with aspects of the present disclosure. FIGS. 2 and 3 show different views illustrating aspects of the system 100. As shown in FIGS. 1 and 2, the environment includes a user 103 and the system 100, comprising an enclosure 105 and a computing system 107.

The user 103 can be any individual. In some implementations, the user 103 is an athlete, such as a track and field athlete, a football player, a hockey player, or the like. In accordance with aspects of the present disclosure, the user 103 can freely run thru the enclosure 105 untethered while the computing system 107 captures images and data of the user's 103 performance.

As illustrated in FIG. 2, in some implementations, the user 103 can be outfitted with wearable devices that capture biometric and motion data. For example, the wearable devices can include instrumented shoes 109 (e.g., MOTICON SCIENCE INSOLES by MOTICON REGO AG, DE) of, a smartwatch 111, smart glasses 113, instrumented earphones, 115, and a motion capture suit 117. These wearable devices 109-117 can include various sensors, such as accelerometers, gaze-detectors, haptic sensors, thermocouples, barometric pressure sensors, heart rate sensors, blood-oxygen level sensors, blood pressure sensors, and other suitable sensors. Additionally, in some implementations the wearable devices 109-117 can include active or passive reference marks at the user's joints, extremities, or other suitable locations for motion capture and analysis.

The enclosure 105 can be a structure including a running surface 108 on which the user 103 can freely run without being tethered. In some implementations, the enclosure 105 comprises a substantially cylindrical shape forming a tunnel enclosing the running surface 108, which extends along the long axis of the cylinder. The cylindrical shape may be circular, rectangular, pentagonal, hexagonal, or other suitable geometry. As illustrated in FIG. 2, the enclosure can be sized to accommodate a single user 103. In some implementations, the internal height of the enclosure is greater than about 7 feet and less than or equal to about 8 feet. In some other implementations, the internal height of the enclosure is greater than about 8 feet and less than or equal to about 9 feet. In some implementations, the internal width of the enclosure is greater than about 6 feet and less than about 9 feet. In some other implementations, the internal width of the enclosure is greater than about 7 feet and less than about 9 feet. In some implementations, length of the enclosure 105 along its long axis is about 10 feet. In some other implementations, the length of the enclosure 105 is between about 40 feet to about 50 feet. In some other implementations, the length of the enclosure 105 is about 100 feet.

As shown in FIG. 1, the enclosure 105 may include ends 119A and 119B, and a sidewall 121. In some implementations, the sidewall 121 includes vertical and horizontal sections forming walls, a ceiling, and a floor of the enclosure 105. In some implementations, the sidewall 121 can be a single wall having a substantially cylindrical shape. In some implementations, the ends 121A and 121B are open such that the runner to run into and out of the enclosure 105. In other implementations, the ends 121A and 121B are closed or are closable to form a substantially closed-space that is isolated from the surrounding environment, such that the environmental conditions (e.g., temperature, pressure, wind) within the enclosure can be controlled by, e.g., a heating, ventilation, and air conditioning system.

The ends 119A and 119B, and the sidewall 121 can be comprised of flexible, semi-rigid, or ridged materials. In flexible or semi-rigid implementations, the ends 119A and 119B, and the sidewall 121 can be made from fabrics, such as PVC (polyvinyl chloride), nylon, and similar materials. In some such implementations, the enclosure 105 can include a frame 147 that supports the ends 119A and 119B, and a sidewall 121. In rigid implementations, the ends 119A and 119B, and the sidewall 121 can be made from wood, steel, aluminum, or other suitable material. In some rigid implementations, the enclosure 105 can be constructed using standardized shipping containers for intermodal freight transport, such as specified by ISO 668:2013 designations 1A, 1B, 1C, or 1D (e.g., 40 feet, 30 feet, 20 feet, and 10 feet).

In some implementations, the enclosure 105 is portable. In some such implementations, a portable enclosure can an air-inflatable structure maintained by a low-pressure fan. Additionally, the ends 119A and 119B, the sidewall 121, and other components can be supported by a frame 147. In such implementations, the enclosure 105 can be inflated and used at a first location, deflated for transport, the frame 147 can be broken down, and the system 100 can be transported. At a second location, the frame 147 can be reconstructed and the enclosure 105 can be re-inflated.

In some other implementations, a portable enclosure 105 can be assembled from one or more modular sections 123A, 123B, 123C, 123D, and 123E, as shown in FIG. 1, that can be connected end-to-end for use at a first location, disconnected for transport, and reconnected for use at the second location. The modular sections 123A-123E can be of the same of different sizes such that the enclosure 105 is reconfigurable to provide different lengths and easy deployment to different facilities of different sizes or requirements. For example, the modular sections can be one or more sizes of standard shipping containers, such as describe above. Accordingly, the length of the enclosure 105 and the running surface 108 can be selectably varied between 10 feet to 100 feet or more. It is understood that different suitable lengths can be used.

FIGS. 2 and 3 illustrate aspects of the interior of the enclosure 105. As shown, implementations of the enclosure 105 can include the running surface 108, sensors 149, cameras 151, display devices 155, lighting devices 159, and audio devices 161. The running surface 108 is capable of supporting the user 103 while running. The running surface 108 is a substantially flat, level plane surrounded by the sidewall 121. In some implementations, the running surface 108 can be comprised of interlinked tiles or from one or more strips that can be rolled-up. In some implementations, the running surface 108 can be made from commercially-available track materials, such as MONDOTRACK® by MONDO S.P.A. of Alba, Italy, or other suitable materials. In some implementations, the running surface 108 can include distance markers placed at increments along the length of the running surface 108. For example, the distance markers may be spaced apart by 2 meters, 5 meters, 10 meters, or more. In some implementations, the distance markers may be placed along the running lane at 10 meters, 20 meters, 30 meters, and 40 meters.

The sensors 149 can include various types of sensor devices. In some implementations, the sensors 147 can include one or more of optical sensors, electromagnetic sensors, ultrasonic sensors, thermocouples, piezoelectric sensors, mechanical sensors, or other suitable sensors for detecting the location, velocity, or acceleration of the user 103. Additionally, in some implementations the sensors 147 include anemometers for measuring wind speed to allow an expert trainer or coach to take into consideration any relevant tailwind or wind resistance that was present during a running performance. Various types of anemometers may be used, such as cup, windmill, hot-wire anemometers and more. Further, in some implementations, the sensors 149 includes haptic sensors 163 connected to the running surface 108 that detect pressure of the user's 103 foot strikes on the upper side of the running surface 108. In implementations, the haptic sensors 163 can be embedded in the running surface 108, or provided as an upper or lower layer of the running surface 108. In implementations, the haptic sensors 163 are distributed over the running surface 108 in one or more force platforms that detect ground reaction force data relevant to human gait and balance. Multiple force platforms may be used to capture ground reaction forces of one or more strides of the user's 103 gait cycle. The reaction forces being measured may be applied downwards towards the ground, and/or upwards away from the ground during different points in the user's 103 running stride.

The cameras 151 can be located in series along the length of the running surface that capture images of user's running form from a variety of perspectives and angles. As illustrated in FIG. 2, the cameras 151 can be mounted on the side and upper locations of the sidewall 121 so as to capture downward and side views of the user 103. Additionally, cameras 151 can be mounted on the ends 119A and 119B to capture front and back views of the user 103. Also, as illustrated in FIG. 3, the cameras 151 can be vertically spaced at incremental distances over the length of the enclosure 105. In some implementations, the cameras are spaced such that their field of views intersect to capture uninterrupted views of the user's 103 running performance. In some implementations, the computing system 107 can process the images from the cameras to stitch the different video feeds so as to provide continuous videos of the running performance over substantially the entire length of the running surface 108 from the different perspectives.

The display devices 155 can be mounted to the sidewall 115. The display devices 155 can be for example liquid crystal display (LCD) display, organic light emitting diode displays (OLED) or other suitable display devices. In some implementations, the display devices 155 can be curved displays or flexible displays. For example, the display devices 155 may have a curved screen following a curvature of the sidewall 115. Additionally, in some implementations flexible displays can be used in combination with fixed flat screens. In some implementations, the display devices 155 can be abutted together along the sidewall 121 over substantially the entire length of the running surface 108 and controlled by the computing system to display cuing images on the display devices 155 that set a pace the user 103 during the running performance. For example, the cueing images can be, a virtual hare, a virtual runner, or images captured from the user's 103 own past performance.

The lighting devices 159 can be placed around the interior of the enclosure 105. The lighting devices can include arrays, or strips of light emitting diodes (LEDs). In some implementations, the lighting devices 159 can be electrically connected and controlled by the computer system 107, which can flash and vary the lighting as desired. For example, the computing system 107 can control the lighting devices 159 to flash at a target running tempo. Additionally, the computing system 107 can control the lighting devices 159 to race an indicator along the length of the running surface 108 indicating a target pace (similar, e.g., to a virtual hare).

The audio devices 161 can include audio speakers and appropriate driving electronics to provide audio cueing to the user 103. For example, the computing system 107 can control the audio devices 161 to generate audio having a beat at a target running tempo. The audio devices 161 may also add to the player's enjoyment of the system 100 by providing music and sound effects designed to enhance and compliment the experience. In FIGS. 2 and 3, the audio devices 161 are shown mounted on the upper portion of the sidewall 115.

It is understood that FIGS. 1-3 illustrate an example of the system 100 and that other implementations are consistent with present with the present disclosure. In some implementations, the locations of the sensors 149, cameras 151, displays devices 155, lighting devices 159, and audio devices 161 can be different, and could include greater or fewer quantities of such devices in different positions and relationships. In some implementations, the size and length of the enclosures 105 may vary such that the enclosure may house a designated running lane of any desired length. It is also understood that the size the enclosure 105 may be increased to provide a second parallel running lane. Further, it us understood that the shape of the enclosure 105 may be a loop to allow for analysis of running performance data collected over longer distances and time intervals.

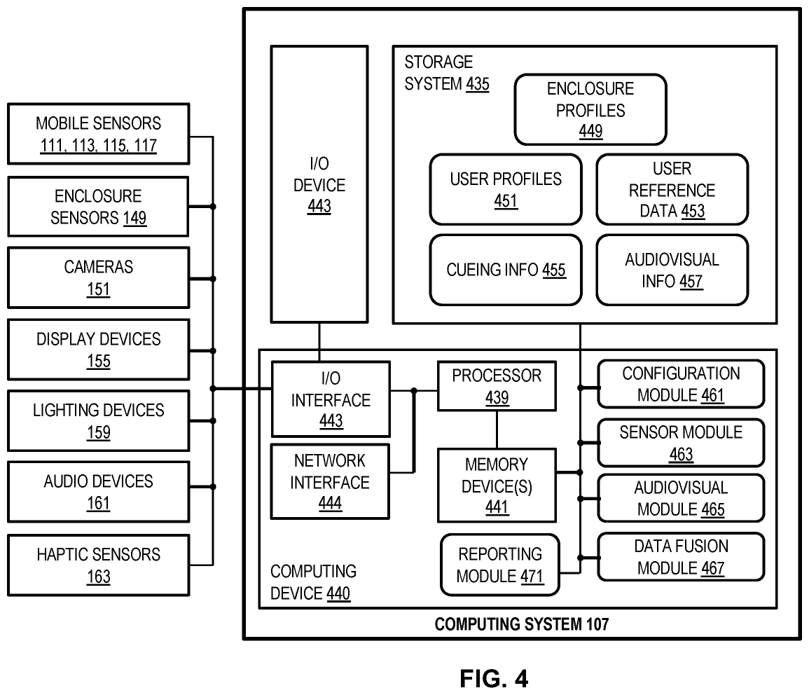

FIG. 4 shows a system block diagram illustrating an example of the computing system 107, which can be the same or similar to that described above. The computing system 107 includes hardware and software that perform the processes and functions disclosed herein. The computing system 107 includes a computing device 430, an input/output (I/O) device 433, and a storage system 435. The I/O device 433 can include any device that enables an individual (e.g., user 103) to interact with the computing device 430 (e.g., a user interface) and/or any device that enables the computing device 430 to communicate with one or more other computing devices using any type of communications link. The I/O device 433 can be, for example, a touchscreen display, pointer device, keyboard, etc.

The storage system 435 can comprise a computer-readable, non-volatile hardware storage device that stores information and program instructions. For example, the storage system 435 can be one or more flash drives and/or hard disk drives. In accordance with aspects of the present disclosure, the storage system 435 can store enclosure profiles 449, user profiles 451, user reference data 453, cueing information 455, and audiovisual information 457. The enclosure profiles 449 can include information describing predetermined arrangements of enclosures (e.g., enclosure 105) corresponding to different predetermined lengths. The user profiles 453 can include information describing users, including a user identification, name, login information, and physical information. The user reference data 453 can include information recorded from past running sessions, as well as associated analysis, reports, and training information. The cueing information 455 can include data for providing running cues a feedback to the user. The audiovisual information 457 can include data for providing lighting and music cues and effects.

In implementations, the computing device 430 includes one or more processors 439 (e.g., microprocessor, microchip, or application-specific integrated circuit), one or more memory devices 441 (e.g., random-access memory (RAM) and read-only memory (ROM)), one or more I/O interfaces 443, and one or more network interfaces 145. The memory device 441 can include a local memory (e.g., a RAM and a cache memory) employed during execution of program instructions. Additionally, the computing device 430 includes at least one communication channel 432 (e.g., a data bus) by which it communicates with the I/O device 433 and the storage system 435. The processor 439 executes computer program instructions (e.g., an operating system and/or application programs), which can be stored in the memory device 441 and/or storage system 435.

The processor 439 can also execute computer program instructions of a configuration module 461, a sensor module 463, an audiovisual module 465, a data fusion module 467, and a reporting module 471. The configuration module 461 can include program instructions for setting up the enclosure 105 to accommodate different lengths and other customizations based on the enclosure profiles 449 and parameters received from operators via the I/O device 443. The sensor module 463 can include program instructions for receiving, conditioning, and storing information from the mobile sensors 111, 113, 115, and 117, the enclosure sensors 149, the cameras 151, and the haptic sensors 163. The audiovisual module 465 can include program instructions for controlling the display devices 155, the lighting devices 159, and the audio devices 161 based on the audiovisual information 457 and inputs received from operators via the I/O device 443. The data fusion module 467 can include program instructions for combining and synchronizing data received from the mobile sensors 111, 113, 115, and 117, the enclosure sensors 149, the cameras 151, and the haptic sensors 163. Fusing the data can include stitching together overlapping image streams from cameras 151 having a same perspective to form continuous videos of the running performance. The reporting module 471 can generate a report of running performance combining associating images representing the sensor data and the videos in a time-synchronized presentation according to a predefined schema. In some implementations, the reporting module 471 may include comparison data from previous running performances stored in the user reference data 453.

It is noted that the computing device 430 can comprise any general-purpose computing article of manufacture capable of executing computer program instructions installed thereon (e.g., a personal computer, server, etc.). However, the computing device 430 is only representative of various possible equivalent-computing devices that can perform the processes described herein. To this extent, in implementations, the functionality provided by the computing device 430 can be any combination of general and/or specific purpose hardware and/or computer program instructions. In each implementation, the program instructions and hardware can be created using standard programming and engineering techniques, respectively.

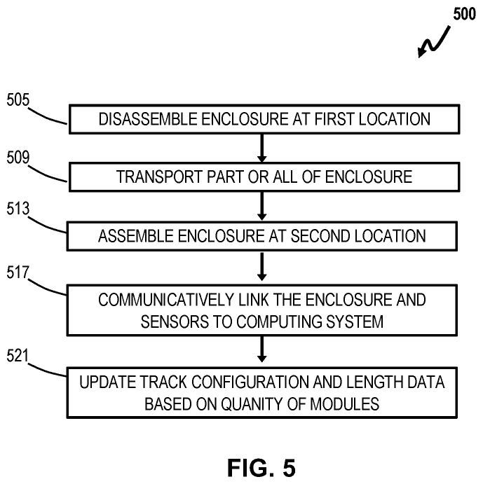

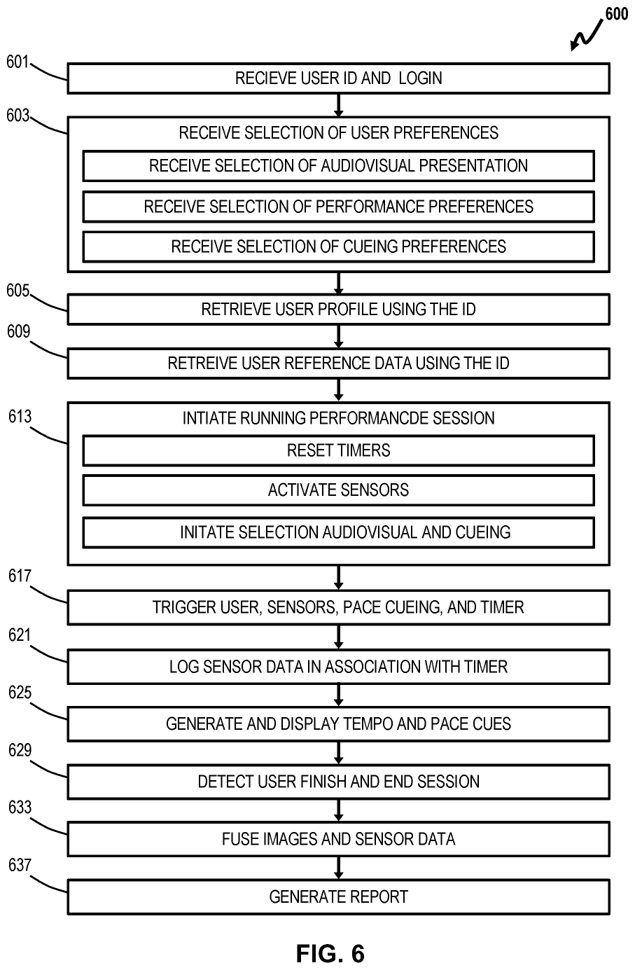

The flow diagrams in FIGS. 5 and 6 illustrate examples of the functionality and operation of possible implementations of systems, methods, and computer program products according to various implementations consistent with the present disclosure. Each block in the flow diagrams of FIGS. 5 and 6 can represent a module, segment, or portion of program instructions, which includes one or more computer executable instructions for implementing the illustrated functions and operations. In some alternative implementations, the functions and/or operations illustrated in a particular block of the flow diagram can occur out of the order shown in FIGS. 5 and 6. For example, two blocks shown in succession can be executed substantially concurrently, or the blocks can sometimes be executed in the reverse order, depending upon the functionality involved. It will also be noted that each block of the flow diagram and combinations of blocks in the block can be implemented by special purpose hardware-based systems that perform the specified functions or acts, or combinations of special purpose hardware and computer instructions.

FIG. 5 illustrates a process 500 of transporting an enclosure in accordance with some implementations of the present disclosure. At block 505, the process 500 can include disassembling the enclosure (e.g., enclosure 105) at a first location. In some implementations, disassembling the enclosure can include removing ends of the enclosure (e.g., ends 119A and 119B), deflating an air-inflatable sidewall (e.g., sidewall 121). Disassembling can also include disassembling a frame (e.g., frame 147) supporting the sidewalls of the enclosure. In other implementations, disassembling the enclosure can include disconnecting two or more modular sections of the enclosure (e.g., sections 123A-123E).

At block 505, the process 500 can include transporting some or all of the enclosure disassembled at block 505. At block 509, the process can include assembling the enclosure at a second location. In some implementations, assembling the enclosure includes reassembling the frame, re-inflating the sidewall, and reconnecting the ends. In some other implementations, assembling the enclosure can include connecting two or more modular sections of the enclosure.

At block 517, the process 500 can include communicatively linking sensors, cameras, display devices, lighting devices, and audio devices to a computer (e.g., computing system 107) at the second location. At block 521, the process 500 can include updating track configuration information based on the quantity of modular sections at block 517, the sensors included in the enclosure, and the display devices, lighting devices, and audio devices mounted in the enclosure.

FIG. 6 illustrates a process 600 of capturing a running performance using a computing system (e.g., system 107) in accordance with some implementations of the present disclosure. At block 601, the computing system can receive a user's identification and login information via an I/O device of a computing system (e.g., I/O device 443 of computing system 107). At block 603, the computing system can receive selections of the user's preferences for a session via the I/O device. Receiving the user preferences can include receiving a selection of an audiovisual presentation for the running session. Receiving the user preferences can also include receiving a selection of performance preferences. Receiving the user preferences can also include receiving a selection of cueing preferences.

At block 605, the computing system can retrieve a user profile (e.g., user profile 451) using the identification information received at block 601. At block 609, the computing system can retrieve user reference data (e.g., user reference data 453) using the identification information received at block 601 and the user profile information retrieved at block 605.

At block 613, the computing system can initiate the session for the user's running performance. Initiating the session can include resetting a timer, activating the sensors, and initiating the audiovisual and cueing routines corresponding to the user's selections at block 603 and the user profile retrieved at block 605.

At block 617, the computing system can trigger the user, sensors, audiovisual routine, cueing routine, and timer. Triggering the user can signal start of the running performance. At block 621, the computing system can log data obtained from the sensors and the cameras in synchronization with data from the timer. In implementations, the computing system can record images of the user's form when running through the enclosure from a two or more perspective. For example, the cameras may record the user running through the enclosure from the front, rear, and one or more side view. The recording can include detecting and capturing markers on the running surface, as well as marks at the user's joints, extremities, or other suitable locations usable for motion capture and analysis.

Additionally, at block 621, the computing device can log data from wearable sensors on the user (e.g., mobile sensors 111, 113, 115, 117), the sensors mounted in the enclosure (e.g., enclosure sensors 149), and the haptic sensors (e.g., haptic sensors 163) in the running surface. As noted above, in some implementations, the haptic sensors can be formed as force platforms that detect ground reaction forces as the user runs over the force platforms and time intervals at which force is being applied to and removed from the platform.

At block 625, the computing system can generate and display cues based on the data logged at block 621. The cues can be presented using the display devices, the lighting devices, and the audio devices. For example, the cues can include sound and lights indicating tempo and pace, a virtual hare, a virtual runner, or images of past performances. At block 629, the computing system detects the user finishing the running performance and ends the session. In some implementations, the computing system automatically detects the finish based on an output of one of the sensors at a finish line of the running surface.

At block 633, the computing system can fuse the image and sensor data collected during the session. Fusing the data can include stitching together overlapping image streams recorded by cameras 151 having a same perspective to form a continuous video of the running performance. At block 637, the computing system can generate a report of the running session. In some implementations, generating the report includes automatically associating the stitched video with data obtained from the sensors based on time using a predefined schema. In some implementations, the reporting module may include comparison data from previous running performances stored in the user reference data.

The present disclosure is not to be limited in terms of the particular implementation described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is also to be understood that the terminology used herein is for the purpose of describing examples of implementations and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to implementations containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” In addition, where features or aspects of the disclosure are described in terms of Markush groups, those skilled in the art will recognize that the disclosure is also thereby described in terms of any individual member or subgroup of members of the Markush group.

1. A system for determining a running performance of a user, the system comprising:
a tunnel;
a length of running surface disposed in the tunnel;
a plurality of sensors disposed along the length of the running surface;
a processor; and
computer-readable data storage device storing program instructions that, when executed by the processor, control the system to log data of the running performance received from the sensors.

2. The system of claim 1, wherein:
the tunnel is a closable space configured to isolate an interior of the tunnel from an environment surrounding the tunnel.

3. The system of claim 1, wherein the tunnel is portable.

4. The system of claim 3, wherein the tunnel is comprised of modular sections.

5. The system of claim 3, wherein the tunnel is inflatable.

6. The system of claim 1, wherein:
the tunnel comprises a substantially cylindrical shape; and
the running surface extends along a long axis of the tunnel.

7. The system of claim 6, wherein:
a height of the tunnel is less than or equal to about 9 feet, and
a length of the tunnel is less than or equal to about 100 feet.

8. The system of claim 1, wherein:
the system further comprises one or more wearable devices, the one or more wearable devices including one or more mobile sensors; and
the program instructions further control the system to:
log data of the running performance received from the one or more wearable sensors; and
combine the data of the running performance received from the sensors with the data of the running performance received from the one or more wearable sensors.

9. The system of claim 8, wherein the one or more wearable sensors are configured to capture biometric data and motion data.

10. The system of claim 1, wherein:
the system further comprises audio devices, display devices, and lighting devices; and
the program instructions further control the system to, based on the data of the running performance, provide audiovisual cues using the audio devices, the display devices, and the lighting devices.

11. The system of claim 10, wherein:
the audiovisual cues indicate a target running pace.

12. The system of claim 10, wherein:
the audiovisual cues comprise time-synchronized recordings of previous running performances.

13. A method for determining a running performance of a user, the method comprising:
initiating a tunnel for the running performance;
triggering the user and a plurality of sensors;
logging image data and sensor data received from the plurality of sensors;
detecting a completion of the running performance; and
generating a report of the running performance, the report including the image data and the sensor data.

14. The method of claim 13, further comprising receiving a selection of user preferences.

15. The method of claim 14, wherein receiving the selection of user preferences comprises receiving a selection of an audiovisual presentation.

16. The method of claim 14, wherein receiving the selection of user preferences comprises receiving a selection of performance preferences.

17. The method of claim 14, wherein receiving the selection of user preferences comprises receiving a selection of cueing preferences.

18. The method of claim 14, wherein the triggering further comprises triggering pace cueing based on the user preferences.

19. The method of claim 18, further comprising generating the pace cueing and tempo cueing along a length of the tunnel.

20. The method of claim 13, wherein the method further comprises fusing the image data and the sensor data.
